(19) United States
(12) Patent Application Publication
(10) Pub. No.: US 2026/0345958 A1
(43) Pub. Date: Nov. 5, 2026

(54) GUIDEWIRE COUPLING DEVICE

(71) Applicant: BIOTEQUE CORPORATION, Taipei City (TW)

(72) Inventors: CHIH-HSIEN CHIEN, Taipei City (TW); SHENG-HUNG YU, Taipei City (TW); JEN-CHIH LIN, Taipei City (TW); CHING-YI HSIEH, Taipei City (TW); ZONG-MING LU, Taipei City (TW); YANG-HU CHEN, Taipei City (TW)

(21) Appl. No.: 19/195,068

(22) Filed: Apr. 30, 2025

(51) Int. Cl. A61M 25/09

(52) U.S. Cl. CPC A61M 25/09; A61M 2025/09125

(57) ABSTRACT

A guidewire coupling device comprises a connecting ring, a catheter-connection cylinder, and a main body assembly. The connecting ring has a first opening and a second opening. The catheter-connection cylinder has a rotating end, a connecting end, and a first channel. The rotating end is mounted through the first opening. The first channel communicates with the connecting end and the rotating end. The main body assembly is mounted through the connecting ring and includes a docking end, a coupling end, and a second channel. The docking end is mounted through the second opening and on the rotating end. The catheter-connection cylinder is rotatable with respect to the docking end. The second channel communicates with the docking end, the coupling end, and the first channel. The guidewire coupling device is capable of coupling with the catheter and other instruments, and the catheter is rotatable all-around without affecting other instruments.

### BACKGROUND

### 1. Field of the Invention

The present invention relates to a medical device, especially to a multi-functional guidewire coupling device that may be applied to manipulate the catheter sleeved on the guidewire.

### 2. Description of the Prior Arts

The guidewire is a high-precision medical instrument, which is usually applied to medical fields such as cardiac catheterization, angiogram, and vascular intervention. The guidewire is capable of moving within the blood vessels which are narrow or tortuous for guiding the catheter or other medical instruments to a precise site. To be more precise, in order to bring the catheter to move smoothly in the blood vessels with the guidewire, the guidewire would protrude by a length with respect to an end portion of the catheter to lead the catheter in direction. The user usually needs to twist the guidewire or rotate the catheter during the process of moving the guidewire and the catheter to a certain site, such that the guidewire or the catheter may move smoothly in the narrow channels.

The conventional guidewire manipulating device may be fixed on a guidewire for the user to apply torque on the guidewire by rotating the manipulating device, helping the guidewire advance in the channels. However, the conventional guidewire manipulating device can only be provided for applying torque on the guidewire, but additional hands are still needed for rotating, moving, and fixing the catheter. Therefore, additional personnel or tools may be acquired to fix the catheter during the surgery to facilitate the moving of the guidewire with respect to the catheter, and that causes inconvenience to the surgery; if the catheter can be directly connected to the guidewire manipulating device, the aforementioned issue may be improved.

In addition, when the guidewire is in use, the guidewire may need to be coupled with another instrument to obtain further treatments such as washing the catheter, and each of the instruments is coupled via a coupling device. If the coupling devices could be combined with the guidewire manipulating device, the numbers of the devices applied in a surgery may be reduced, and the user may obtain convenience during the surgery without location changes.

However, if the coupling device, the guidewire manipulating device, and the catheter are directly combined together, the instruments coupled to the coupling device would be brought to rotate when the user is rotating the catheter, and as the instruments include wires or tubes, the instruments may be twisted together, affecting the surgery accordingly.

To overcome the shortcomings, the present invention provides a guidewire coupling device to mitigate or obviate the aforementioned problems.

### SUMMARY

The main objective of the present invention is to provide a guidewire coupling device that can couple with a catheter and other instruments, and the user may control the catheter via the guidewire coupling device without affecting the instruments.

The guidewire coupling device comprises a connecting ring, a catheter-connection cylinder, and a main body assembly. The connecting ring has a first opening and a second opening located on two opposite surfaces respectively. An inner space is formed within the connecting ring. The inner space, the first opening, and the second opening spatially communicate with each other. The catheter-connection cylinder is rotatably mounted on the connecting ring, and the catheter-connection cylinder has a rotating end, a connecting end, and a first channel. The rotating end is mounted through the first opening and extends to the inner space. The connecting end extends along a direction away from the rotating end. The first channel is formed through the catheter-connection cylinder, and the first channel spatially communicates with the connecting end and the rotating end. The main body assembly is mounted through the connecting ring, and the main body assembly includes a docking end, a coupling end, and a second channel. The docking end is mounted through the second opening and mounted on the rotating end of the catheter-connection cylinder, and the catheter-connection cylinder is capable of rotating with respect to the docking end. The coupling end is opposite to the docking end. The second channel is formed through the main body assembly and spatially communicates with the docking end and the coupling end, and the second channel and the first channel communicate with each other.

The advantage of the guidewire coupling device in this disclosure is multi-functionality: the guidewire coupling device is movable freely along the guidewire, and helps couple the catheter and other instruments with the guidewire, and the user can control the catheter via the guidewire coupling device. The user can rotate the catheter all-around via the guidewire coupling device without affecting the instruments coupled, preventing the tubes or the wires from getting twisted, and thus provides convenience to manipulation of the guidewire.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a first embodiment of a guidewire coupling device in accordance with the present invention;

FIG. 2 shows the guidewire coupling device of FIG. 1 viewed from another angle;

FIG. 3 is an exploded view of the guidewire coupling device in FIG. 1;

FIG. 4 shows the guidewire coupling device of FIG. 3 viewed from another angle;

FIG. 5 is a cross-sectional side view of the guidewire coupling device in FIG. 1;

FIG. 6 is a partial enlarged cross-sectional side view of the guidewire coupling device in FIG. 1, showing the docking end mounted on the rotating end;

FIG. 7 is a perspective view of the connecting ring of the guidewire coupling device in FIG. 1;

FIG. 8 is a perspective view of the catheter-connection cylinder of the guidewire coupling device in FIG. 1;

FIG. 9 is a perspective view of the main cylinder of the guidewire coupling device in FIG. 1;

FIG. 10 is a perspective view of the fixing cylinder of the guidewire coupling device in FIG. 1;

FIG. 11 is a perspective view of a second embodiment of the guidewire coupling device in accordance with the present invention, shown without the fixing unit;

FIG. 12 is an exploded view of the guidewire coupling device in FIG. 11;

FIG. 13 is a perspective view of the main body assembly of the guidewire coupling device in FIG. 11;

FIG. 14 is a cross-sectional side view of the guidewire coupling device in FIG. 11;

FIG. 15 is another perspective view of the guidewire coupling device in FIG. 11, shown connecting with the fixing unit;

FIG. 16 is a cross-sectional side view of the guidewire coupling device in FIG. 15; and

FIG. 17 is a side view of the first embodiment of the guidewire coupling device in FIG. 1, shown coupling with a catheter, a guidewire, and an instrument.

### DETAILED DESCRIPTION

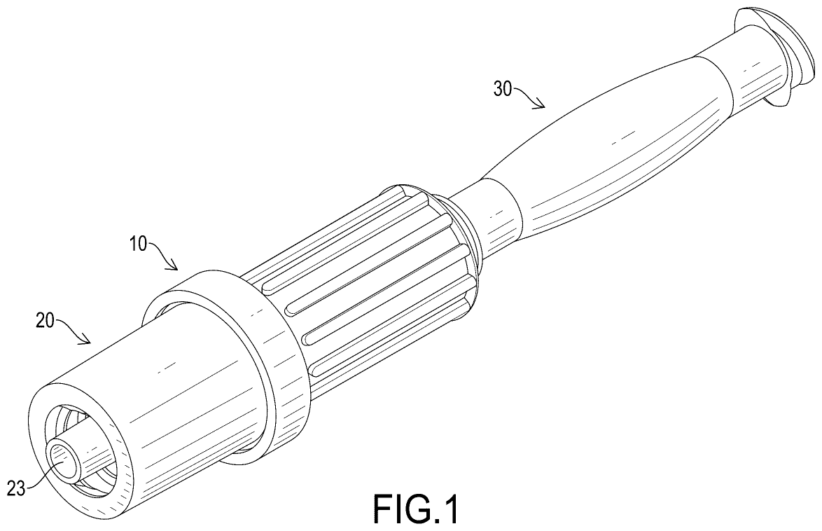

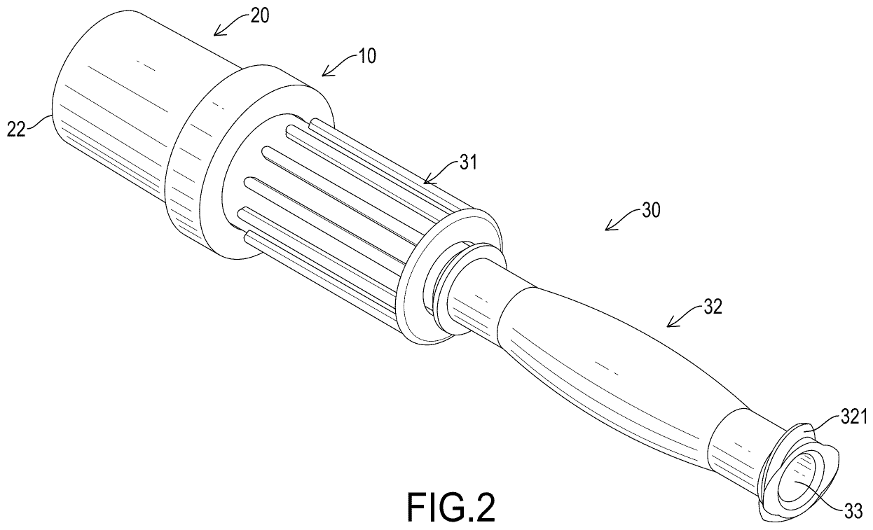

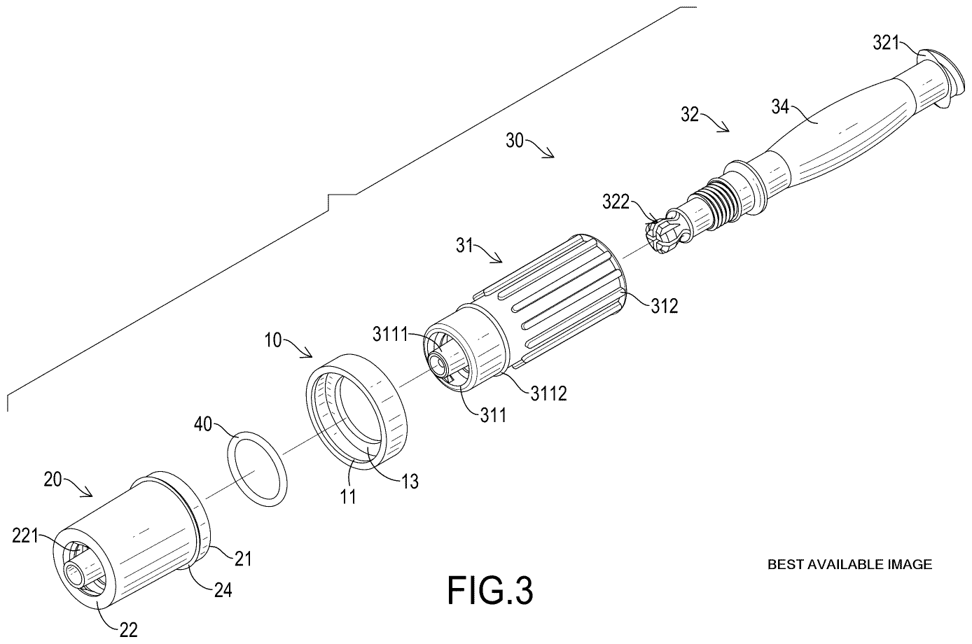

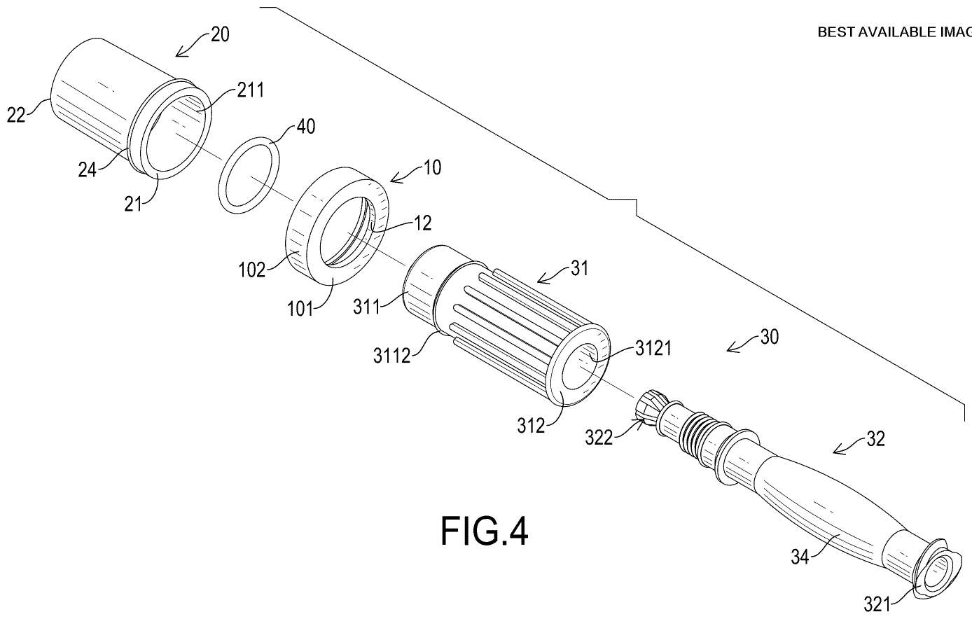

With reference to FIGS. 1 to 4 and 17, a first embodiment of a guidewire coupling device in accordance with the present invention, which is configured to mount a guidewire B, comprises a connecting ring 10, a catheter-connection cylinder 20, a main body assembly 30, and a filling ring 40.

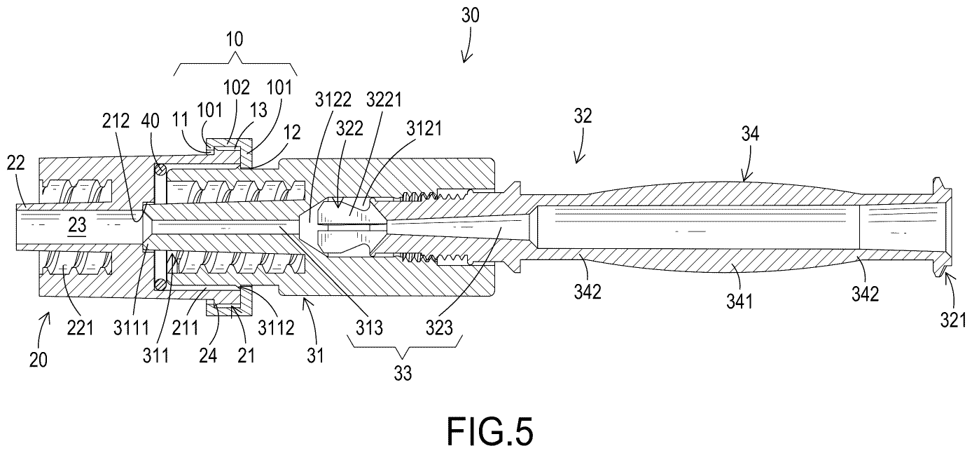

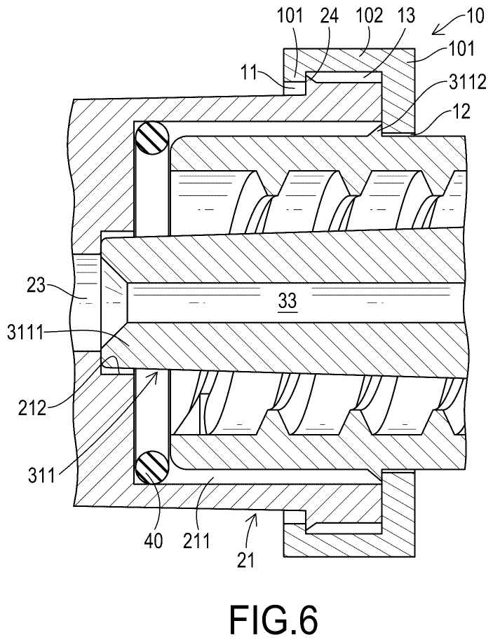

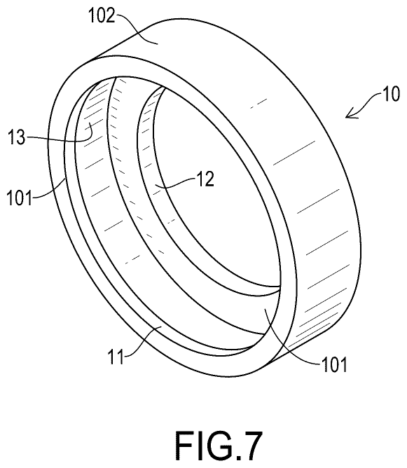

With reference to FIGS. 1 and 3 to 7, a first opening 11 and a second opening 12 are respectively located on two opposite surfaces of the connecting ring 10, and an inner space 13 is formed in the connecting ring 10, and the inner space 13, the first opening 11, and second opening 12 spatially communicate with each other. To be more precise, the connecting ring 10 includes two plates 101 and an annular wall 102, wherein the two plates 101 are spaced apart from each other and the annular wall 102 is connected to a peripheral part of each one of the two plates 101, such that the inner space 13 is formed among the two plates 101 and the annular wall 102. The first opening 11 and the second opening 12 are formed on the two plates 101 respectively, and thus the inner space 13 spatially communicates with the first opening 11 and the second opening 12, but it is not limited thereto.

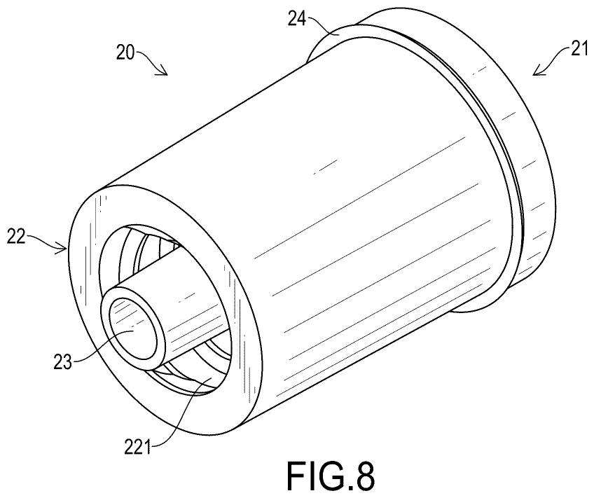

With reference to FIGS. 1, 3 to 5, 8, and 17, the catheter-connection cylinder 20 is rotatably mounted on the connecting ring 10. The catheter-connection cylinder 20 in this embodiment is cylindrical and thus has a rotating end 21 and a connecting end 22 which are opposite to each other, and a first channel 23 is formed through the catheter-connection cylinder 20 and spatially communicates with the rotating end 21 and the connecting end 22. When the guidewire coupling device is mounted on the guidewire B, the guidewire B would be disposed through the first channel 23, and the user may connect a catheter A to the catheter-connection cylinder 20.

To be more precise, the rotating end 21 of the catheter-connection cylinder 20 is mounted through the first opening 11 of the connecting ring 10 and extends into the inner space 13. In this embodiment, the rotating end 21 has a mounting recess 211 and a first external annular rib 24. The mounting recess 211 is recessed on an end surface of the rotating end 21, and a limiting recess 212 is further recessed from a basal part of the mounting recess 211, but it is not limited thereto; in another embodiment, the rotating end 21 may not have the mounting recess 211 or the basal part of the mounting recess 211 may not have the limiting recess 212.

The first external annular rib 24 protrudes from an outer annular surface of the rotating end 21 and is located within the inner space 13 of the connecting ring 10. The first external annular rib 24 abuts a periphery part around the first opening 11 of the connecting ring 10, such that the catheter-connection cylinder 20 is rotatable with respect to the connecting ring 10 without separating from the connecting ring 10.

The connecting end 22 extends along a direction away from the rotating end 21 and is configured to connect the catheter A. To be more precise, an outer annular recess 221 is recessed from an end surface of the connecting end 22 in this embodiment; the outer annular recess 221 circumferentially surrounds the first channel 23, and a partition is located between the first channel 23 and the outer annular recess 221, thereby the outer annular recess 221 receiving and connected with the catheter A which is sleeved on the guidewire B. Furthermore, an inner wall surface of the outer annular recess 221 in this embodiment forms threads, thereby connecting the catheter A via threading together, but it is not limited thereto.

The first channel 23 is formed through the catheter-connection cylinder 20 and spatially communicates with the connecting end 22 and the rotating end 21. In this embodiment, the first channel 23 forms an opening on a basal part of the limiting recess 212, and the first channel 23 spatially communicates with the mounting recess 211 via the limiting recess 212, but it is not limited thereto.

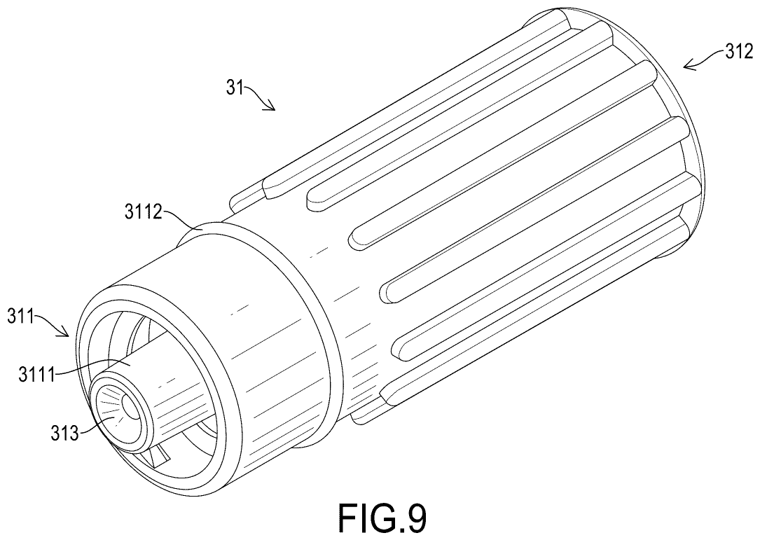

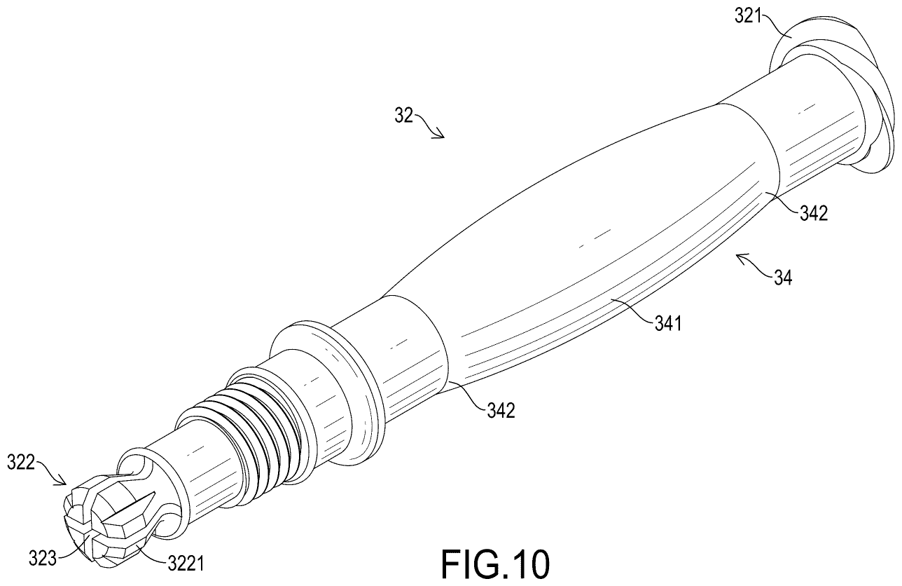

With reference to FIGS. 1, 3 to 5, 9, and 10, the main body assembly 30 includes a docking end 311, a coupling end 321, and a second channel 33. The docking end 311 and the coupling end 321 are two opposite ends of the main body assembly 30 respectively. The second channel 33 is formed through the main body assembly 30, spatially communicating with the docking end 311 and the coupling end 321. The docking end 311 of the main body assembly 30 is mounted through the second opening 12 of the connecting ring 10 and mounted on the rotating end 21 of the catheter-connection cylinder 20; the catheter-connection cylinder 20 is rotatable with respect to the docking end 311, and the second channel 33 and the first channel 23 spatially communicate with each other.

To be more precise, in this embodiment, the main body assembly 30 may include a main cylinder 31 and a fixing cylinder 32. The docking end 311 is at the main cylinder 31 and the coupling end 321 is at the fixing cylinder 32.

The main cylinder 31 is disposed through the connecting ring 10, the docking end 311 is an end of the main cylinder 31, and the main cylinder 31 further includes a receiving end 312 and a first sub-channel 313. To be more precise, the docking end 311 and the receiving end 312 are two opposite ends of the main cylinder 31, and the first sub-channel 313 is formed through and spatially communicates with the docking end 311 and the receiving end 312. The docking end 311 is disposed through the second opening 12 of the connecting ring 10 and extends within the inner space 13. The docking end 311 is mounted in the mounting recess 211 of the rotating end 21 of the catheter-connection cylinder 20, and the docking end 311 is rotatable with respect to the catheter-connection cylinder 20.

The receiving end 312 is another end of the main cylinder 31 which is opposite to the docking end 311, and a fixing recess 3121 is recessed from an end surface of the receiving end 312. The fixing recess 3121 includes a fixing section 3122, and an inner diameter of the fixing section 3122 gradually decreases along a direction from the receiving end 312 to the docking end 311. The first sub-channel 313 is part of the second channel 33, and the first sub-channel 313 is formed in and spatially communicates with the docking end 311 and a basal part of the fixing recess 3121, and thereby the first sub-channel 313 and the fixing recess 3121 spatially communicate with each other.

In this embodiment, the docking end 311 may further include an inner cylindrical wall 3111 and a second external annular rib 3112. The inner cylindrical wall 3111 circumferentially surrounds the second channel 33, and an end of the inner cylindrical wall 3111 is disposed in the limiting recess 212 of the mounting recess 211 of the catheter-connection cylinder 20, and thereby the first channel 23 may be aligned with the second channel 33 to avoid shifting during the catheter-connection cylinder 20 rotating with respect to the main body assembly 30, but it is not limited thereto.

The second external annular rib 3112 protrudes from an outer annular surface of the docking end 311 and is located within the inner space 13 of the connecting ring 10; the second external annular rib 3112 abuts a periphery part around the second opening 12 of the connecting ring 10, thereby preventing the main body assembly 30 separating from the second opening 12, but it is not limited thereto.

The fixing cylinder 32 is disposed in the fixing recess 3121 of the main cylinder 31 and is movable with respect to the main cylinder 31. The coupling end 321 is an end of the fixing cylinder 32 and the fixing cylinder 32 further includes a clamping end 322 and a second sub-channel 323. In this embodiment, the fixing cylinder 32 is mounted in the fixing recess 3121 of the main cylinder 31 in a threading manner, thus being easy for the user to adjust a displacement of the fixing cylinder 32 with respect to the fixing recess 3121, but it is not limited thereto. The clamping end 322 is another end of the fixing cylinder 32 which is opposite to the coupling end 321, and the clamping end 322 is located in the fixing section 3122 of the fixing recess 3121. The second sub-channel 323 is also part of the second channel 33. The second sub-channel 323 is formed through two opposite ends of the fixing cylinder 32 and penetrates the clamping end 322, and an inner diameter of the second sub-channel 323 at the clamping end 322 is adjustable. The first sub-channel 313 and the second sub-channel 323 spatially communicate with each other and together form the second channel 33.

In this embodiment, the clamping end 322 comprises multiple clamping pieces 3221 which are spaced apart from each other, and the clamping pieces 3221 surround the second sub-channel 323. The clamping pieces 3221 are spaced apart along a radial direction and a circumferential direction of the second sub-channel 323, and the clamping pieces 3221 are elastic. The clamping pieces 3221 may be deformed along a force direction when the clamping pieces 3221 are pressed by the force. Hence, when the fixing cylinder 32 moves with respect to the fixing recess 3121, the clamping pieces 3221 would be compressed by an inner annular wall of the fixing section 3122, such that the clamping pieces 3221 deform inwardly along the radial direction, further decreasing the inner diameter of the second sub-channel 323 at the clamping end 322 and clamping the guidewire B, and a position of the guidewire B is fixed, but a configuration of the clamping end 322 is not limited thereto.

The main body assembly 30 may further include a handle portion 34. The handle portion 34 is located between the docking end 311 and the coupling end 321, and in this embodiment, the handle portion 34 is disposed on the fixing cylinder 32. The handle portion 34 has a middle site 341 and two end sites 342. The middle site 341 is connected to and located between the two end sites 342, and an outer diameter of the handle portion 34 gradually decreases from the middle site 341 to the two end sites 342, but it is not limited thereto; in another embodiment, the main body assembly 30 may not have the handle portion 34.

The filling ring 40 is disposed between the docking end 311 and a basal part surface of the mounting recess 211. The filling ring 40 fills the space between the docking end 311 and a basal part surface of the mounting recess 211, thereby stabilizing a position of the main body assembly 30 relative to the catheter-connection cylinder 20 in an axial direction, but it is not limited thereto.

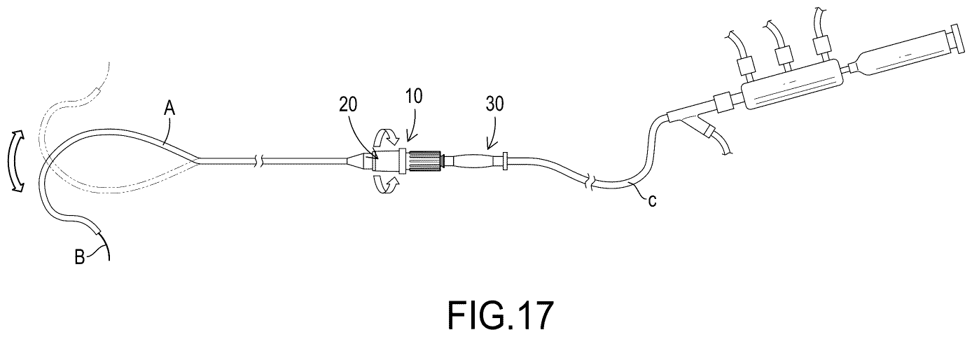

With reference to FIGS. 1, 4 to 6, and 17, when the guidewire coupling device in this embodiment is in use, the user may put the guidewire B into the first channel 23 via the connecting end 22 of the catheter-connection cylinder 20. The guidewire B is then put into the second channel 33 of the main body assembly 30 via the first channel 23, and then comes out from the guidewire coupling device via the coupling end 321 of the main body assembly 30; therefore, the guidewire coupling device is movable with respect to the guidewire B along the extending direction of the guidewire B. Furthermore, the user may connect the catheter A with the connecting end 22 of the catheter-connection cylinder 20. To be more precise, in this embodiment, the user may connect the catheter A with the outer annular recess 221 by threading to the outer annular recess 221 via a connecting sleeve (not shown in the drawings) which is set with the catheter A. Due to the catheter A being fixed to the catheter-connection cylinder 20 and the guidewire B being movable through the guidewire coupling device, the guidewire B is movable with respect to the catheter A. When the user is going to fix the protruding length of an end of the guidewire B protruding from the catheter A, the user may move the fixing cylinder 32 with respect to the main cylinder 31, such that the clamping pieces 3221 of the clamping end 322 would be compressed by the inner annular wall of the fixing section 3122 of the fixing recess 3121, and the inner diameter of the part of the second channel 33 within the fixing section 3122 decreases. Hence, the clamping pieces 3221 are capable of clamping the guidewire B and fixing a relative position between the guidewire B and the guidewire coupling device, i.e., fixing the protruding length of the end of the guidewire B protruding from the catheter A. In addition, the coupling end 321 of the main body assembly 30 is provided for coupling with other instruments, tubes, or wires (such as the instrument C in FIG. 17), the configuration of the coupling end 321 may be altered according to need.

After setting up, to rotate the catheter A, the user may grab the handle portion 34 of the main body assembly 30 with one hand, and rotate the catheter-connection cylinder 20 with the other hand, such that the catheter A can be brought to rotate all-around. Due to connecting with the main body assembly 30 via the connecting ring 10, the catheter-connection cylinder 20 is rotatable with respect to the connecting ring 10, and the connecting ring 10 is also rotatable with respect to the main body assembly 30, and since the main body assembly 30 couples with the catheter-connection cylinder 20 by disposing in the mounting recess 211 of the catheter-connection cylinder 20, the catheter-connection cylinder 20 is freely rotatable with respect to the main body assembly 30 without bringing the main body assembly 30 to rotate together, thereby preventing the instrument C, connected to the main body assembly 30, from twisting. In addition, the user may rotate the main body assembly 30 to further apply torque to the guidewire B when the user wants to twist the guidewire B.

Furthermore, in this embodiment, the inner cylindrical wall 3111 of the docking end 311 of the main body assembly 30 is disposed in the limiting recess 212 formed on the basal part of the mounting recess 211, and thereby when the catheter-connection cylinder 20 rotates with respect to the main body assembly 30, the docking between the first channel 23 and the second channel 33 would not be broken; moreover, since the first external annular rib 24 of the catheter-connection cylinder 20 abuts the periphery part of the connecting ring 10 around the first opening 11, and the second external annular rib 3112 of the main body assembly 30 abuts the periphery part of the connecting ring 10 around the second opening 12, the catheter-connection cylinder 20 and the main body assembly 30 would not separate from the connecting ring 10 and the catheter-connection cylinder 20 can rotate steadily. In addition, the filling ring 40 disposed between the docking end 311 and the basal part of the mounting recess 211 is capable of stabilizing the relative position of the catheter-connection cylinder 20 and the main body assembly 30 along the axial direction.

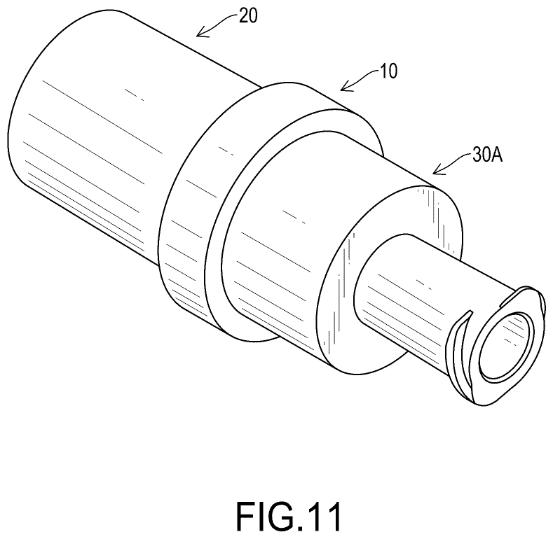

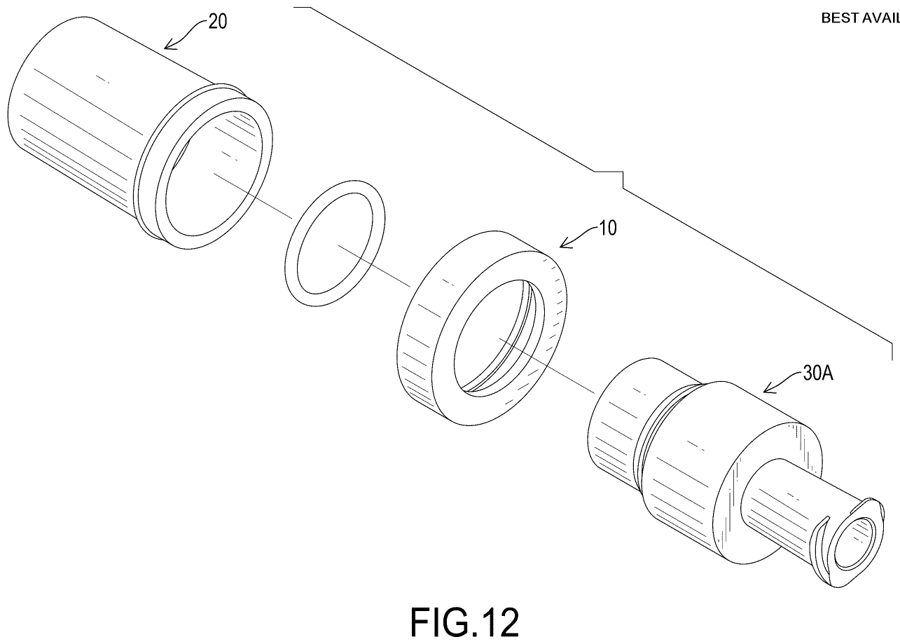

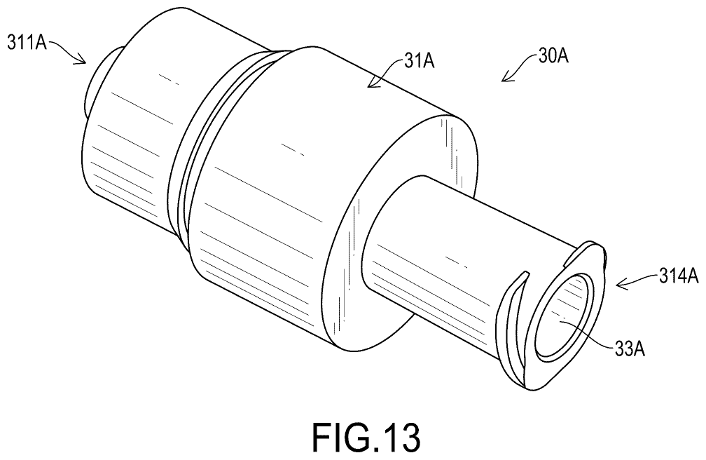

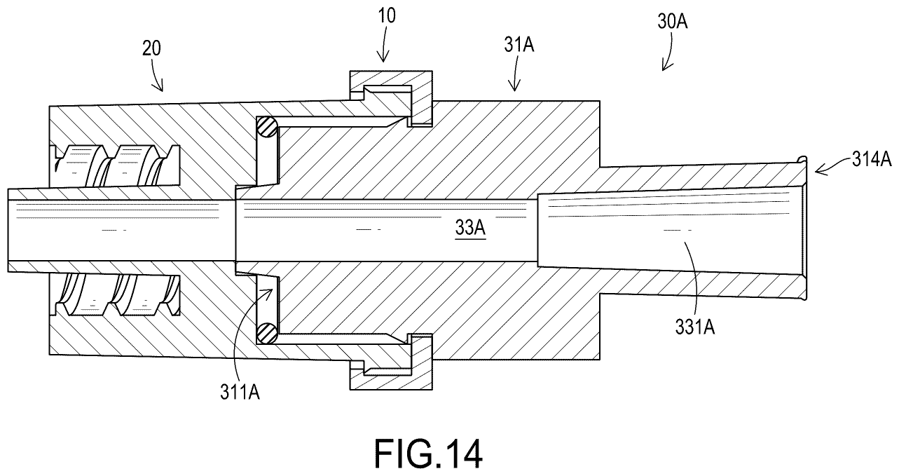

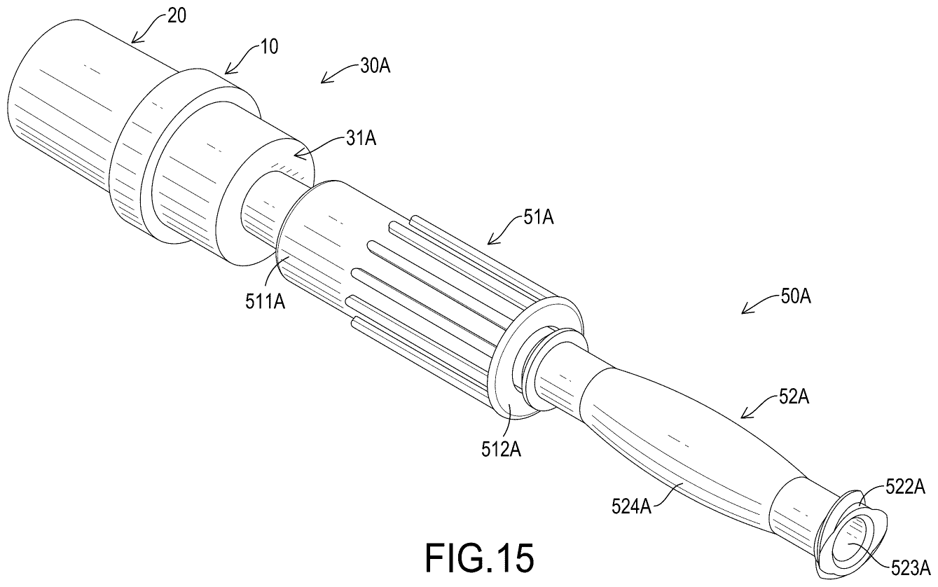

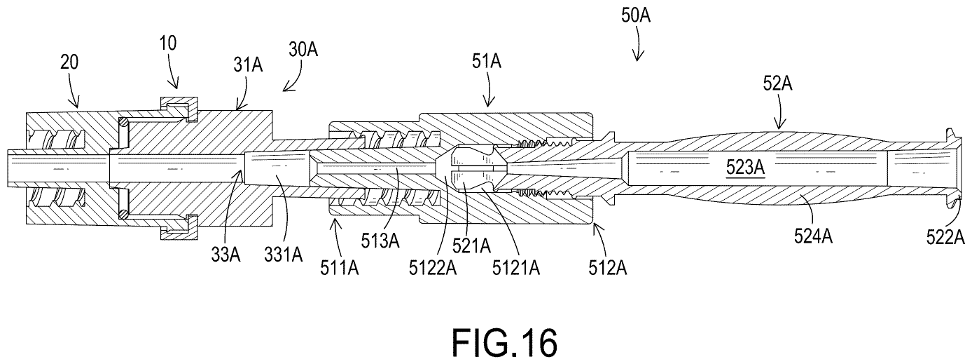

With reference to FIGS. 11 to 14, a second embodiment of the guidewire coupling device in accordance with the present invention comprises a connecting ring 10, a catheter-connection cylinder 20, and a main body assembly 30A. The second embodiment is similar to the first embodiment, but a main difference from the first embodiment is the main body assembly 30A. In short, in the second embodiment, the functions of the main body assembly 30A in fixing the length of the guidewire and connecting with the catheter-connection cylinder 20 are split. In the second embodiment, the main body assembly 30A has a main cylinder 31A, and the guidewire coupling device in this embodiment may further comprise a fixing unit 50A as shown in FIGS. 15 and 16.

The main cylinder 31A in the second embodiment is similar to the main cylinder 31 in the first embodiment but without the fixing recess 3121. To be more precise, in the second embodiment, the main cylinder 31A is mounted through the connecting ring 10, and the docking end 311A and the coupling end 314A are the two opposite ends of the main cylinder 31A. The second channel 33A is located in the main cylinder 31A. The second channel 33A further includes a receiving section 331A, and an inner diameter of the receiving section 331A gradually decreases along a direction from the coupling end 314A to the docking end 311A.

The fixing unit 50A is connected to the coupling end 314A of the main cylinder 31A. In this embodiment, the fixing unit 50A comprises a first sub-cylinder 51A and a second sub-cylinder 52A.

The first sub-cylinder 51A has a first end 511A, a second end 512A, a fixing recess 5121A, and a third channel 513A. The first end 511A and a second end 512A are two opposite ends of the first sub-cylinder 51A, and the first end 511A is disposed through the receiving section 331A, thereby the first sub-cylinder 51A being connected to the main cylinder 31A. To be more precise, the first end 511A is inserted into and tight fit with the receiving section 331A, such that the first sub-cylinder 51A is fixed onto the main cylinder 31A without separating. The fixing recess 5121A is recessed from the second end 512A and includes a fixing section 5122A. An inner diameter of the fixing section 5122A gradually decreases along a direction from the second end 512A to the first end 511A. The third channel 513A is formed through and spatially communicates with the first end 511A and a basal part of the fixing recess 5121A.

The second sub-cylinder 52A is movably mounted through the first sub-cylinder 51A and has a clamping end 521A, a joining end 522A, and a fourth channel 523A. The clamping end 521A and the joining end 522A are two opposite ends of the second sub-cylinder 52A, and the clamping end 521A is disposed through the fixing section 5122A of the fixing recess 5121A. The fourth channel 523A is formed through and spatially communicates with the clamping end 521A and the joining end 522A of the second sub-cylinder 52A, and an inner diameter of a part of the fourth channel 523A at the clamping end 521A is adjustable. The third channel 513A spatially communicates with the fourth channel 523A and the second channel 33A, and thereby the guidewire may be disposed within the fourth channel 523A, the third channel 513A, and the second channel 33A.

The relationship between the first sub-cylinder 51A and the second sub-cylinder 52A in the second embodiment is similar to the relationship between the main cylinder 31 and the fixing cylinder 32 in the first embodiment. The fixing recess 5121A of the first sub-cylinder 51A corresponds to the fixing recess 3121 of the main cylinder 31 in the first embodiment, and the clamping end 521A of the second sub-cylinder 52A corresponds to the clamping end 322 of the fixing cylinder 32 in the first embodiment. In addition, in this embodiment, the handle portion 524A is disposed on the second sub-cylinder 52A, but it is not limited thereto.

The operating way of the second embodiment is similar to the first embodiment, and the main difference is that the function of fixing the protruding length of the guidewire with respect to the catheter is provided by the fixing unit 50A, and other instruments may be coupled to the joining end 522A of the fixing unit 50A in addition to the coupling end 314A of the main cylinder 31A, and thus the second embodiment is capable of providing more diverse connection with the instruments. The user may directly set the fixing unit 50A on the guidewire for applying torque to and manipulating the guidewire or coupling with other instruments if there is no need to sleeve the catheter on the guidewire. On the other hand, the user may remove the fixing unit 50A and couple the instruments to the coupling end 314A of the main cylinder 31A if there is no need to fix the protruding length of the guidewire with respect to the catheter, such that the user can still freely rotate the catheter connected to the catheter-connection cylinder 20 all-around without affecting the instruments coupled to the main cylinder 31A. Besides, the coupling end 314A in the second embodiment may be configured to couple with a conventional guidewire manipulator/torque applier, but it is not limited thereto.

The guidewire coupling device in this disclosure is movable along the guidewire, and capable of connecting the catheter, coupling other instruments, and fixing the protruding length of the guidewire with respect to the catheter; the user can rotate the catheter all-around via the guidewire coupling device without affecting other instruments coupled with the guidewire coupling device, preventing the wires or tubes from twisting together. Besides, the catheter-connection cylinder 20 in this disclosure may smoothly rotate with respect to the main body assembly 30, 30A, so the first channel 23 and the second channel 33 would stably spatially communicate with each other without affecting the guidewire. To sum up, the guidewire coupling device in this disclosure has multiple functions, which may reduce the complexity of the connection of the catheter and/or the instruments in a surgery, reduce the manpower for supporting and holding the instruments, and provide an operating site for manipulating the guidewire and the catheter, and thus brings convenience to the surgery requiring high precision and concentration.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A guidewire coupling device comprising:
a connecting ring having a first opening and a second opening located on two opposite surfaces respectively; an inner space formed within the connecting ring, and the inner space, the first opening, and the second opening spatially communicating with each other;
a catheter-connection cylinder rotatably mounted on the connecting ring, and the catheter-connection cylinder having:
a rotating end mounted through the first opening and extending into the inner space;
a connecting end extending along a direction away from the rotating end; and
a first channel formed through the catheter-connection cylinder, and the first channel spatially communicating with the connecting end and the rotating end; and
a main body assembly mounted through the connecting ring, and the main body assembly including:
a docking end mounted through the second opening and mounted on the rotating end of the catheter-connection cylinder, and the catheter-connection cylinder capable of rotating with respect to the docking end;
a coupling end being opposite to the docking end; and
a second channel formed through the main body assembly and spatially communicating with the docking end and the coupling end, and the second channel and the first channel communicating with each other.

2. The guidewire coupling device as claimed in claim 1, wherein the main body assembly comprises:
a main cylinder disposed through the connecting ring, and the docking end being an end of the main cylinder; the main cylinder including:
a receiving end being another end of the main cylinder opposite to the docking end;
a fixing recess recessed from the receiving end and including a fixing section, and an inner diameter of the fixing section gradually decreasing along a direction from the receiving end to the docking end; and
a first sub-channel formed in and spatially communicating with the docking end and a basal part of the fixing recess; and
a fixing cylinder movably disposed through the fixing recess, and the coupling end being an end of the fixing cylinder; the fixing cylinder including:
a clamping end being another end of the fixing cylinder opposite to the coupling end, and the clamping end disposed in the fixing section of the fixing recess; and
a second sub-channel formed through the fixing cylinder and penetrating the clamping end, and an inner diameter of the second sub-channel at the clamping end being adjustable;
wherein, the first sub-channel and the second sub-channel spatially communicate with each other and together form the second channel.

3. The guidewire coupling device as claimed in claim 1, wherein the main body assembly comprises:
a main cylinder disposed through the connecting ring, and the docking end and the coupling end being two opposite ends of the main cylinder respectively, and the second channel located within the main cylinder;
the second channel further comprising a receiving section, and an inner diameter of the receiving section gradually decreasing along a direction from the coupling end to the docking end.

4. The guidewire coupling device as claimed in claim 3, wherein the guidewire coupling device further comprises:
a fixing unit connected to the coupling end of the main cylinder, and the fixing unit including:
a first sub-cylinder having:
a first end and a second end being two opposite ends of the first sub-cylinder, and the first end disposed through the receiving section;
a fixing recess recessed from the second end; the fixing recess including a fixing section, and an inner diameter of the fixing section gradually decreasing along a direction from the second end to the first end; and
a third channel formed through and spatially communicating with the first end and a basal part of the fixing recess; and
a second sub-cylinder movably mounted through the first sub-cylinder, and the second sub-cylinder having:
a clamping end and a joining end being two opposite ends of the second sub-cylinder respectively, and the clamping end disposed in the fixing section of the fixing recess; and
a fourth channel formed through and spatially communicating with the clamping end and the joining end of the second sub-cylinder, and an inner diameter of the fourth channel at the clamping end being adjustable;
wherein, the third channel spatially communicates with the fourth channel and the second channel.

5. The guidewire coupling device as claimed in claim 1, wherein the catheter-connection cylinder further comprises:
a first external annular rib protruding from an outer annular surface of the rotating end and located within the inner space of the connecting ring, and the first external annular rib abutting a periphery part around the first opening of the connecting ring.

6. The guidewire coupling device as claimed in claim 2, wherein the catheter-connection cylinder further comprises:
a first external annular rib protruding from an outer annular surface of the rotating end and located within the inner space of the connecting ring, and the first external annular rib abutting a periphery part around the first opening of the connecting ring.

7. The guidewire coupling device as claimed in claim 4, wherein the catheter-connection cylinder further comprises:
a first external annular rib protruding from an outer annular surface of the rotating end and located within the inner space of the connecting ring, and the first external annular rib abutting a periphery part around the first opening of the connecting ring.

8. The guidewire coupling device as claimed in claim 1, wherein the main body assembly further comprises:
a second external annular rib protruding from an outer annular surface of the docking end and located within the inner space of the connecting ring, and the second external annular rib abutting a periphery part around the second opening of the connecting ring.

9. The guidewire coupling device as claimed in claim 6, wherein the main body assembly further comprises:
a second external annular rib protruding from an outer annular surface of the docking end and located within the inner space of the connecting ring, and the second external annular rib abutting a periphery part around the second opening of the connecting ring.

10. The guidewire coupling device as claimed in claim 7, wherein the main body assembly further comprises:
a second external annular rib protruding from an outer annular surface of the docking end and located within the inner space of the connecting ring, and the second external annular rib abutting a periphery part around the second opening of the connecting ring.

11. The guidewire coupling device as claimed in claim 1, wherein a mounting recess is recessed from the rotating end, and the docking end is mounted in and is rotatable with respect to the mounting recess.

12. The guidewire coupling device as claimed in claim 9, wherein a mounting recess is recessed from the rotating end, and the docking end is mounted in and is rotatable with respect to the mounting recess.

13. The guidewire coupling device as claimed in claim 10, wherein a mounting recess is recessed from the rotating end, and the docking end is mounted in and is rotatable with respect to the mounting recess.

14. The guidewire coupling device as claimed in claim 11, wherein:
a limiting recess is recessed from a basal part of the mounting recess, and the first channel forms an opening at a basal part of the limiting recess; and
the docking end comprises an inner cylindrical wall which circumferentially surrounds the second channel, and an end of the inner cylindrical wall is disposed in the limiting recess.

15. The guidewire coupling device as claimed in claim 12, wherein:
a limiting recess is recessed from a basal part of the mounting recess, and the first channel forms an opening at a basal part of the limiting recess; and
the docking end comprises an inner cylindrical wall which circumferentially surrounds the second channel, and an end of the inner cylindrical wall is disposed in the limiting recess.

16. The guidewire coupling device as claimed in claim 13, wherein:
a limiting recess is recessed from a basal part of the mounting recess, and the first channel forms an opening at a basal part of the limiting recess; and
the docking end comprises an inner cylindrical wall which circumferentially surrounds the second channel, and an end of the inner cylindrical wall is disposed in the limiting recess.

17. The guidewire coupling device as claimed in claim 1, wherein an outer annular recess is recessed from the connecting end and circumferentially surrounds the first channel.

18. The guidewire coupling device as claimed in claim 15, wherein an outer annular recess is recessed from the connecting end and circumferentially surrounds the first channel.

19. The guidewire coupling device as claimed in claim 16, wherein an outer annular recess is recessed from the connecting end and circumferentially surrounds the first channel.

20. The guidewire coupling device as claimed in claim 1, wherein, the main body assembly comprises a handle portion located between the docking end and the coupling end; the handle portion comprises:
a middle site and two end sites; the middle site connected to and located between the two end sites, and an outer diameter of the handle portion gradually decreasing from the middle site to the two end sites.
